(19) United States
(12) Patent Application Publication
(10) Pub. No.: US 2021/0080358 A1
(43) Pub. Date: Mar. 18, 2021

(54) SYSTEMS AND METHODS FOR COLLECTING, PROCESSING, OPTIMIZING, AND PREPARING STEM CELLS FOR FURTHER USE

(71) Applicant: Elite IP, LLC, Johnstown, CO (US)

(72) Inventors: Edward Jeffrey Donner, Fort Collins, CO (US); Lucanus Steven Koldewyn, Johnstown, CO (US); Christopher Thomas Donner, Fort Collins, CO (US); Ryan Dregalla, Lyons, CO (US); Kristopher Buchanan, Fort Collins, CO (US); William Buchanan, Fort Collins, CO (US); Robert Mitchell Clark, Centennial, CO (US)

(73) Assignee: Elite IP, LLC, Johnstown, CO (US)

(21) Appl. No.: 17/006,778

(22) Filed: Aug. 28, 2020

Related U.S. Application Data
(60) Provisional application No. 62/893,032, filed on Aug. 28, 2019.

(51) Int. Cl. G01N 1/08; G01N 1/28; C12N 5/0775; A61K 35/28

(52) U.S. Cl. CPC G01N 1/08; A61K 35/28; C12N 5/0663; G01N 1/286

(57) ABSTRACT

A system for collecting and processing a sample of an enriched population of mesenchymal stem cells in a matrix of body tissue in which the sample is separated into smaller segments to optimize the ability of the system to organize the stem cells into small working clusters with tissue. The tissue clusters are separated into optimized groupings for further processing and use in medical procedures.

### CROSS REFERENCE TO RELATED APPLICATIONS

This application claims the benefit of U.S. Provisional Application No. 62/893,032, filed Aug. 28, 2019, the entire disclosure of which is incorporated herein by reference.

### FIELD OF THE INVENTION

The present invention relates generally to the field of medical and laboratory apparatus and methods of using the same. More specifically, the present invention relates to methods and apparatus for the extraction of tissue, cells, and other constituents from an enclosed body cavity such as a bone marrow cavity. Specifically, the present invention relates to methods and apparatus for increasing the efficiency of harvesting and processing mesenchymal stem cells, other biologic material, and physiological fluids for use in medical diagnostic, treatment, or research applications.

### BACKGROUND OF THE INVENTION

Mesenchymal stem cells, which for purposes of brevity will be referred to herein as (“MSC” or “MSC's”), are stromal or connective tissue cells which are found primarily in umbilical cord blood and bone marrow. Unlike hematopoietic stem cells (“HSC”) which contribute to the formation of red blood cells, MSC can differentiate into a variety of cell types such as bone, cartilage, muscle, and fat cells. While the cell differentiation phenomenon observed in MSC's is not fully understood, medical research has established that MSC and other constituents of bone marrow are remarkably capable of contributing to a patient's healing and tissue regeneration processes following injury or surgery and may be used therapeutically to treat numerous medical conditions.

MSC harvesting and processing cells and physiological fluid may be accomplished by several different conventional methods and apparatus. Exemplary methods and devices in current use involve multiple aspirations of bone marrow cells (BMC) from the patient's posterior iliac crest. Access to the internal cancellous bone, bone marrow and bone marrow blood residing inside the outer cortical bone layer of the ilium is achieved by creating an aperture in the cortical bone with a sharp instrument such as a trocar. An aspiration needle, for example, a Jamshidi bone marrow biopsy needle, is then inserted through the aperture into the interior trabecular compartments within the crest where the MSC are found in their highest concentrations. However, a significant problem associated with conventional methods for harvesting and processing cells and physiological fluid (including the aspiration of bone marrow from bone for producing stem cell concentrate) is that a less than desirable amount, type or quality of cells or physiological fluid may be harvested and subsequently processed while still maintaining cell viability. Substantial problems with respect to procedure efficiency, duration and desirable amounts, concentrations, purity, and viability of cells or biologic material remain unresolved.

Accordingly, a need exists for an improved system and method and apparatus for the harvesting and processing of cancellous bone, bone marrow and bone marrow blood for producing mesenchymal stem cell concentrate and other physiological fluids which addresses the combination of problems not solved by the prior art.

### SUMMARY OF THE INVENTION

The stated problems and other needs in the art as apparent from the foregoing background may be addressed in accordance with the methods and apparatus of the present invention as set forth in various embodiments disclosed herein.

In one implementation of the present invention, a system for producing and maximizing the yield and viability of harvested tissue, by way of example, an enriched stem cell product, is disclosed which includes a bone core harvesting instrument, a modular receptacle or container adapted to receive the harvested bone core and any associated bone marrow, cells, and/or physiological fluid, a securing device or base member adapted to receive and secure the modular receptacle or container, a processing device adapted to process the harvested bone core in the modular receptacle or container, and a treatment syringe and needle.

According to an embodiment, the bone marrow harvesting instrument is a Jamshidi needle (also known as a trocar or stylet) movably deployed within an outer cannula having at least one externally-threaded end, the needle being adapted to create an aperture in the patient's cortical bone structure at a preselected location and to collect tissue, e.g., a bone core having a bony matrix, bone marrow, and bone marrow blood and deposit it into the modular receptacle.

In another embodiment, the processing device is configured to be secured by the securing device or base member and interface with the modular receptacle to process the harvested bone marrow, cells, and/or physiological fluid.

In yet another embodiment, the securing device or base member is adapted to be operatively secured to or mounted on a work surface such as a work or laboratory bench top.

In still another embodiment, the securing device or base member includes a manually powered apparatus configured to provide the mechanical power to process the bone core inside the modular receptacle.

In another embodiment, the securing device or base member includes an electrically or magnetically powered apparatus configured to provide electromechanical power to process the bone core inside the modular receptacle.

In an embodiment, the securing device or base member is configured to automate the processing of the bone core whereby the cells and tissue in the bone core are processed in a controlled manner pursuant to a preselected set of processing parameters.

In another embodiment, the modular receptacle or container is configured to receive and contain a carrier fluid.

In still another embodiment, the processing device includes a morselizing tool portion configured to morselize the bone core into smaller bone core pieces.

In yet another embodiment, the processing device includes a washing or rinsing portion configured to wash or rinse the bone core pieces whereby certain biologic material such as glycosaminoglycan (GAG) may be stripped from the bony matrix of the bone core pieces and put into solution with the carrier fluid.

In another embodiment, the washing portion is adapted to further break down the stripped biologic material into smaller predetermined sized portions.

In still another embodiment, the processing device may perform both the morselizing and the washing of the bone core with a single tool member.

In another embodiment, the single tool member may be actuated in response to preselected program processing parameters to perform the morselizing and the washing of the bone core.

In yet another embodiment, preselected program processing parameters include by way of example and not of limitation, morselizing, the single tooling portion may be located at a different location within the disposable module, operated at a different speed (whether rotationally or linearly reciprocated), or operated for a different duration, pulsed or continuous motion, and the like for the morselizing operation versus for the washing or rinsing operation.

In still another embodiment, the system of the present invention is structured and arranged for a single use, to be disposed of thereafter.

In another embodiment, the system of the present invention is structured and arranged to be sterilized for multiple use.

In an embodiment, a method for harvesting and processing a patient's mesenchymal stem cells is provided which includes inserting a Jamshidi-type needle, having a preselected diameter or size and a cannula extending circumferentially about and along the length thereof into the cortical bone at a preselected location on a patient's skeletal system forming an aperture therein; removing the stylet; advancing the cannula into the patient's bone marrow to obtain a cancellous bone plug or core; aspirating bone marrow material, blood, MSC and MSC-like cells through a cancellous bone plug positioned in the cannula to increase the harvested MSC cell count; and processing the harvested material in the system herein disclosed for producing and maximizing the yield and viability of an enriched stem cell product.

These and other features of the present invention will be apparent from the accompanying description of the invention, drawings, diagrams, and supplemental supporting materials provided herein.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1.A is an isometric view of components of a system for collecting, processing, optimizing, and preparing tissue such as bone core containing MSC harvested from a patient in accordance with an embodiment.

FIG. 1.B is an isometric sectional view of the system of FIG. 1.A having portions removed to illustrate the elements thereof more clearly in accordance with an embodiment.

FIG. 2 is an isometric sectional view of a system for collecting, processing, optimizing, and preparing tissue such as bone core containing MSC harvested from a patient illustrating certain components of the system including a carrier fluid loaded within a first syringe and a harvesting tool for collecting tissue from a patient in accordance with an embodiment of the present invention.

FIG. 3 is a side sectional view of a tissue collecting or harvesting portion of a system for the collecting, processing, optimizing, and preparing stem cells illustrating the initial position of a Jamshidi needle in the system in accordance with an embodiment.

FIG. 4 is a side sectional view of the system of FIG. 3 illustrating a Jamshidi needle containing a bone core positioned in a passageway of a spindle portion of the system up to an internal stop at a distal end of the passageway.

FIG. 5 is a side sectional view of the system of FIG. 4 having the Jamshidi needle removed and a plug reinserted into the spindle passageway.

FIG. 6 is a side sectional view of the view of the system of FIG. 5 in a depressed or closed configuration.

FIG. 7 is a side sectional view of the view of the system of FIG. 6 in an extended or open configuration having a syringe containing a predetermined amount of carrier fluid operatively connected to a proximal end of a spindle element of the system.

FIG. 8 is a side sectional view of the system of FIG. 7 in an extended or open configuration illustrating a plunger of the syringe depressed and the carrier fluid repositioned in a passageway in the spindle and in a chamber in the base member.

FIG. 9 is a side sectional view of the system of FIG. 8 in an extended or open configuration having the syringe removed and the plug reinserted into the spindle passageway.

FIG. 10 is a side sectional view of the system of FIG. 9 in a depressed or closed configuration.

FIG. 11 is a side sectional view of the system of FIG. 10 in a depressed or closed configuration the plug removed from the spindle and a syringe operatively connected to the proximal end of the spindle.

FIG. 12 is a side sectional view of the system of FIG. 11 with the syringe plunger withdrawn and the carrier fluid and processed biologic material solution extracted from the chamber in the base member, through the spindle and into the syringe.

FIG. 13 is an isometric view of a disposable system for collecting, processing, optimizing, and preparing stem cells in accordance with an embodiment.

FIG. 14 is an isometric view of a handle or knob member and cap, spindle and upper morselizing or grinding components of the disposable system of FIG. 13 in accordance with an embodiment.

FIG. 15 is an isometric view of a detachable base member of the disposable system of FIG. 13 in accordance with an embodiment.

FIG. 16 is a top plan view of the upper morselizing or grinding components of the disposable system of FIG. 13 in accordance with an embodiment.

FIG. 17 is a top plan view of the upper morselizing or grinding components of the disposable system of FIG. 13 in accordance with an embodiment.

FIG. 18.A is a side perspective view of a morselizing or grinding component of the system of the present invention in accordance with another embodiment of the present invention.

FIG. 18.B is a bottom plan view of the of morselizing or grinding component of the embodiment of FIG. 18.A.

FIG. 19.A is a side perspective view of a morselizing or grinding component of the system of the present invention in accordance with another embodiment of the present invention.

FIG. 19.B is a bottom plan view of a morselizing or grinding component of the embodiment of FIG. 19.A.

FIG. 20.A is a side perspective view of a morselizing or grinding component of the system of the present invention in accordance with another embodiment of the present invention.

FIG. 20.B is a bottom plan view of a morselizing or grinding component of the embodiment of FIG. 20.A.

FIG. 21 is a bar graph showing the post-processing viability of MSC cells in two samples.

FIG. 22.A are photomicrographs at 2× and at 4× of a first sample of tissue cells taken directly from a culture.

FIG. 22.B are photomicrographs 2× and at 4× of a first sample of tissue cells following a first passage from one vessel to a larger vessel.

FIG. 22. C are photomicrographs at 2× and 4× of a first sample of tissue cells following a second passage to yet a larger vessel.

FIG. 23.A are photomicrographs of a second sample of tissue cells taken directly from a culture.

FIG. 23.B are photomicrographs of a second sample of tissue cells following a first passage from one vessel to a larger vessel.

FIG. 23.C are photomicrographs of a second sample of tissue cells following a second passage from one vessel to a larger vessel.

FIG. 24 is a bar graph showing high density MSC growth post bone core processing.

### DETAILED DESCRIPTION OF THE INVENTION

It should be noted that the present description is by way of illustration only, and that the concepts and examples presented herein are not limited to use or application with any single system or methodology. Hence, while the details of the system and methods described herein are for the convenience of illustration and explanation with respect to the exemplary embodiments, the principles disclosed may be applied to other types of tissue harvesting systems and methods without departing from the scope of the present invention.

Implementations of the present invention involve a system for processing a harvested tissue for preparing a biologic mixture. In particular, the system may include a collecting or harvesting tool for collecting tissue from a patient, a first module adapted to receive tissue from the harvesting tool and to process the tissue, a carrier fluid to receive and transport the tissue, a base module or member operably connected to the first or processing module, the base member being structured and arranged to be either hand-held or to be secured to a work surface or bench, and a collection and delivery apparatus or tool. The harvesting tool may include a tubular component configured to receive the tissue for collection and delivery into the disposable module. The harvesting tool may further include a plunger configured to force the tissue from the tubular component and into the base module. The various components, including both the first or processing module and the base member may be disposable or may be designed and constructed to be sterilized and reused for multiple patients and procedures.

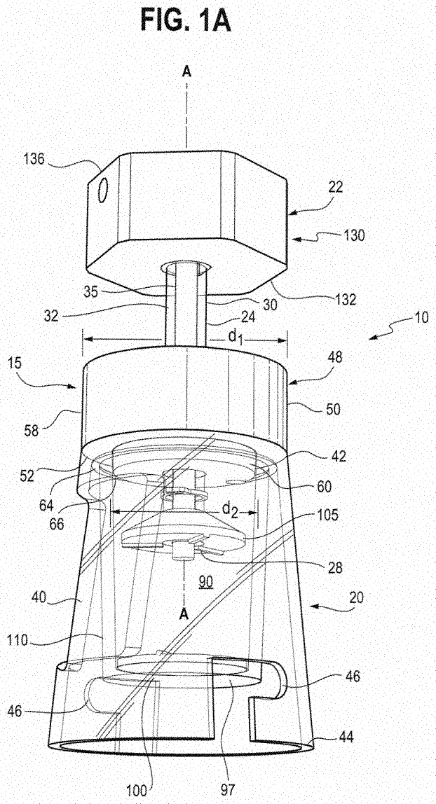

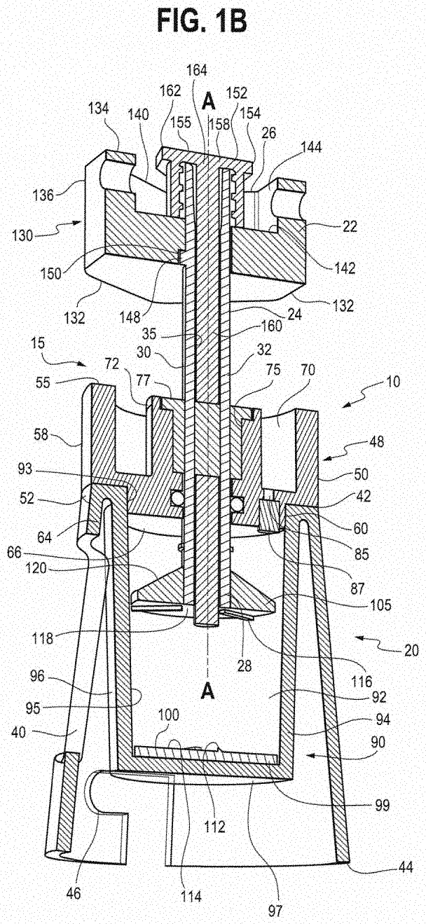

Referring now to FIGS. 1.A and 1.B, a system for collecting, processing, optimizing, and preparing tissue such as bone core containing mesenchymal stem cells (MSC) harvested from a patient is shown generally at 10. The system includes a first or upper module 15 adapted to be operatively connected to and to cooperate with a base member or module 20 for the processing of tissue collected from a human patient for treatment purposes or from an animal for research.

The first module includes a handle or knob 22 adapted to be hand-operated or to be operatively connected to an electrically or magnetically powered apparatus configured to provide electromechanical power to process the harvested tissue such as bone core material and/or MSC following collection. As best viewed in FIG. 1.B, the knob is operatively connected to a spindle 24 at a first end 26 thereof. The spindle further includes a second end 28 and a cylindrical body 30 having a wall portion 32 extending intermediate the first and second ends along axis A-A and defining a coaxially extending cylindrical aperture 35 therein extending coaxially intermediate the ends.

The base member 20 includes a slightly tapered cylindrically shaped body, preferably formed of a transparent material such a clear plastic or a polycarbonate material such as Lexan® to permit observation of the processing operation. The body has a wall portion 40 extending circumferentially about the axis A-A and extending intermediate upper and lower open ends 42, 44, the upper open end having a smaller diameter than the diameter of the lower open end, thus forming an isosceles trapezoidal-shaped cross sectional body configuration as shown in FIG. 1.B. The tapered body enhances the operating stability of the system, particularly if the processing operation is being performed by hand by allowing a technician to grasp the system firmly and to apply a stabilizing downward force with one hand while loading and processing material with the other hand. Alternatively, for use when an electromechanical power source is employed to power the processing operation, the base member includes a plurality of P-shaped apertures or slots 46 formed in the wall 40 and extending upwardly from the lower open end, each of the plurality of slots being adapted to receive a suitable fastener, chuck, or clamping device to secure the system to a work surface.

A closure device, cap, or cover 48 is adapted to fit in removable sealing engagement into the open upper end 42 of the base member 20 in a manner analogous to a stopper or cork being urged into engagement with the end of a bottle or jar. In the embodiment of FIGS. 1.A and 1.B, the cap includes a first cylindrical body member 50 having a diameter d₁ extending coaxially along and circumferentially about axis A-A, a bottom or lower surface 52, a top or upper surface 55, and a side surface 58 extending intermediate the bottom and top surfaces. A second cylindrical body member 60 having a diameter d₂, a sidewall 64 and a bottom surface 66 is operatively connected to the bottom surface 52 of the first cylindrical body member and extends downwardly therefrom into the open upper end of the base member coaxially therewith along axis A-A. The first cylindrical body member 50 further includes a channel 70 formed in the upper surface 55 extending circumferentially about axis A-A forming an upwardly extending stem 72 having a stepped aperture 75 formed therein adapted to receive a bushing 77 supporting the spindle 24. A recess 79 located in the aperture 75 is structured and arranged to receive an O-ring 80 to provide sealing engagement with the spindle 24 extending rotatably therethrough. A second stepped aperture 85 having a filter positioned therein extends intermediate the bottom surface 66 and the channel 70 to vent gases formed in a modular receptacle or container 90 adapted to receive the harvested bone core and any associated bone marrow, cells, and/or physiological fluid, as will be described in greater detail below.

Referring again to FIG. 1.A, the modular receptacle 90 is in the form of a straight-walled cylindrical container 92 having an open upper end 93 adapted to fit in releasable sealing engagement with the second cylindrical body member 60, a cylindrical wall portion 94 having an inner surface 95 and an outer surface 96 extending intermediate the open upper end and a closed bottom portion 97. The closed bottom portion has an inner surface 99. A first or bottom grinding or morselizing tool or plate 100 is operatively connected to the inner surface and is structured and arranged to cooperate with a second or upper grinding or morselizing tool or plate 105 secured to the second end 28 of the spindle 24 to process harvested bone core material. The modular receptacle is formed of a transparent material such as glass or polycarbonate plastic to permit observation of the morselizing operation and has a slight outward wall taper from the bottom to the top to facilitate an injection molding process. However, the angle between the inner surface 95 of the cylindrical wall and the inner surface 99 of the bottom is approximately orthogonal to prevent extrusion of the harvested material from between the upper and lower morselizing tools and up the inner surface during processing. A viewing aperture 110 is formed in the wall portion 40 of the base member to facilitate observation of the morselizing process.

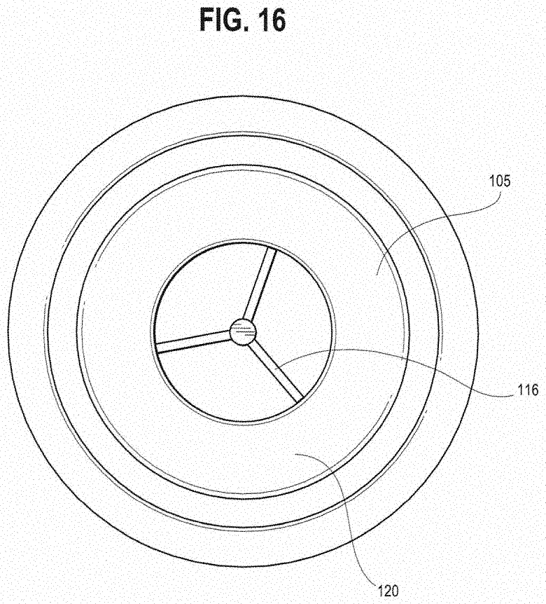

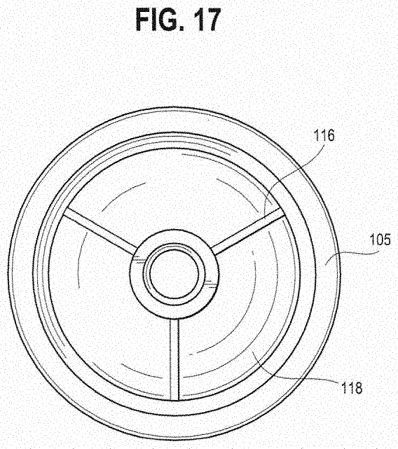

The lower grinding plate 100 includes a plurality of radially outwardly extending fingers or vanes 112 formed integrally with or operatively connected to an upper surface 114 thereof. Similarly, as best shown in FIG. 17, the upper morselizing or grinding tool 105 includes a plurality of radially outwardly extending fingers or vanes 116 formed integrally with or operatively connected to a lower surface 118 thereof. While in the embodiment shown, the vanes are oriented radially perpendicularly to a circumferential peripheral edge of the grinding plate, the vanes may be oriented at other angles depending upon the direction of rotation of the upper grinding plate. Moreover, the upper grinding or morselizing tool 105 incudes a top or upper surface 120 (FIG. 16) which is sloped or pitched to facilitate runoff of the harvested material during processing operations.

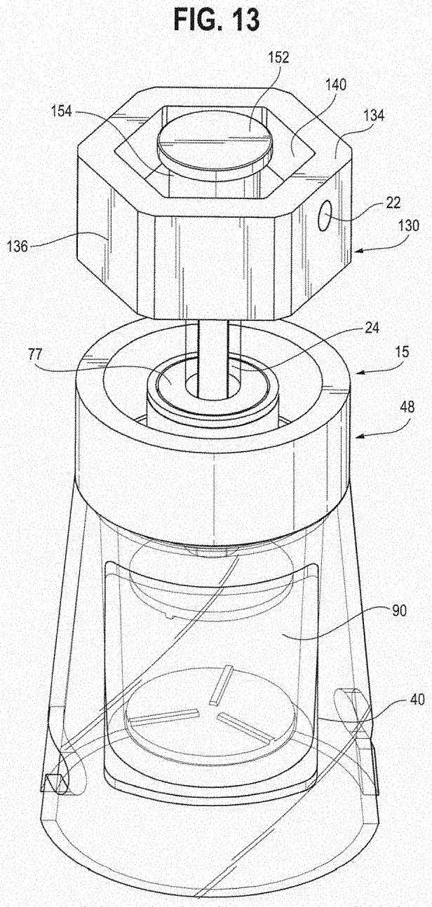

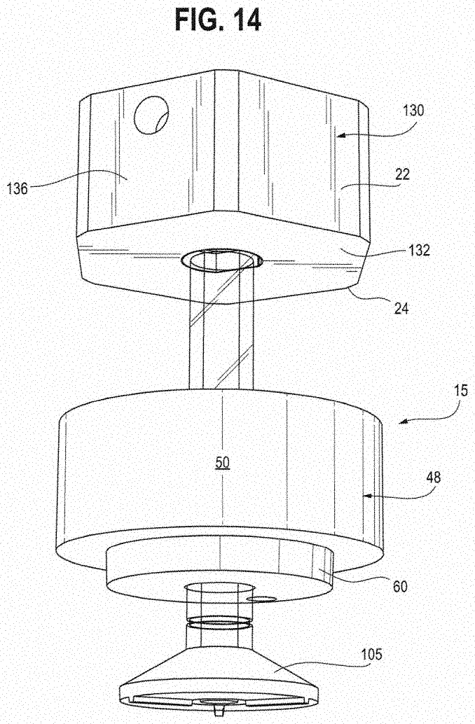

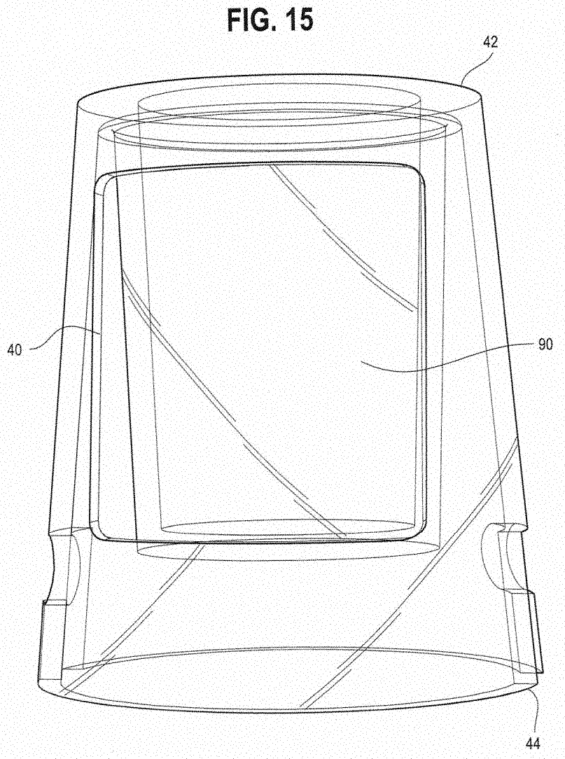

Referring again to FIGS. 1.A and 1.B and as shown in greater detail in FIGS. 13 and 14, first or upper module 15 is described in greater detail. For purposes of illustration, the knob 22 and spindle 24 are shown in the extended or open configuration. In the embodiment shown, the knob includes a polygon shaped body 130 having a lower or bottom surface 132, an upper or top surface 134, and a plurality of side portions 136 extending intermediate the upper and lower surfaces. However, it is to be understood that the knob 22 may have other configurations such as oval or cylindrical and have scored or knurled surfaces to minimize slipping in one's hands while performing processing operations without departing from the scope of the present invention.

A cylindrically shaped recess 140 is formed in the upper surface 134 extending circumferentially about axis A-A. The recess includes a bottom surface 142 and an inner surface 144 extending intermediate the bottom surface 142 of the recess and the upper surface 134 of the knob. An aperture 146 is formed concentrically about axis A-A extending through the bottom surface 132 of the knob and is adapted to rotatably and slidably receive the spindle 24, whereby the knob, spindle and upper morselizing or grinding plate may be controllably moved from an extended open configuration to a closed or depressed configuration. The spindle is releasably secured to the knob via a pin member 148 extending radially outwardly from wall portion 32 of the spindle and received in a notch or recess 150 formed in the aperture 146. A plug 152. A Luer connection 155 is formed on the first end 26 of the spindle and is adapted to provide a connection to a syringe or a filter and an interface for an automated an electrically or magnetically powered apparatus drive tool configured to provide electromechanical power to process the harvested tissue. A plug 158 having an elongate body 160 is releasably positioned in the coaxially extending cylindrical aperture 35 in the spindle and is adapted to prevent samples of harvested material from being trapped therein following injection or from being forced upwardly through the aperture during processing. The plug includes a grip or knob 162 operatively connected to a first end 164 of the plug, the grip having a female Luer connector 166 formed therein and adapted to fit in locking engagement with Luer connection 155 formed on the first end 26 of the spindle.

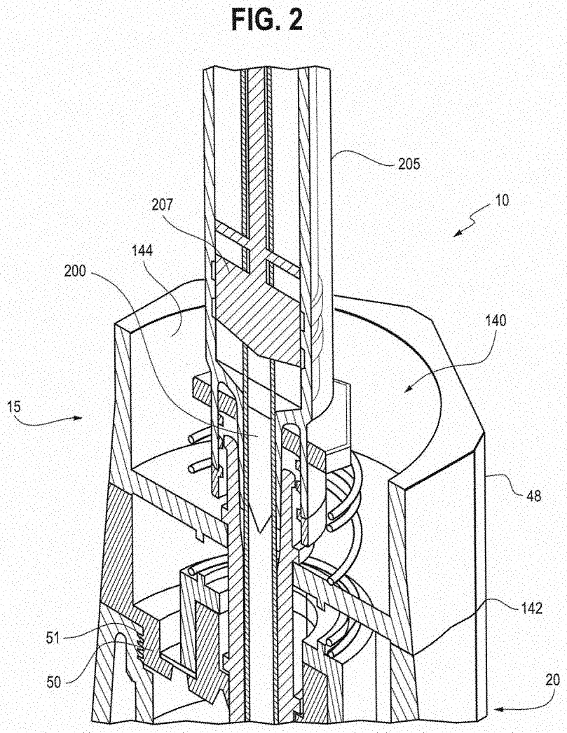

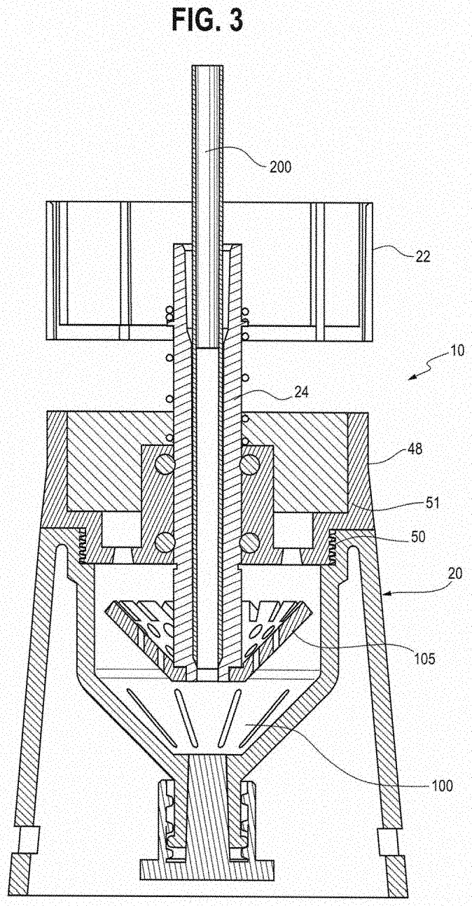

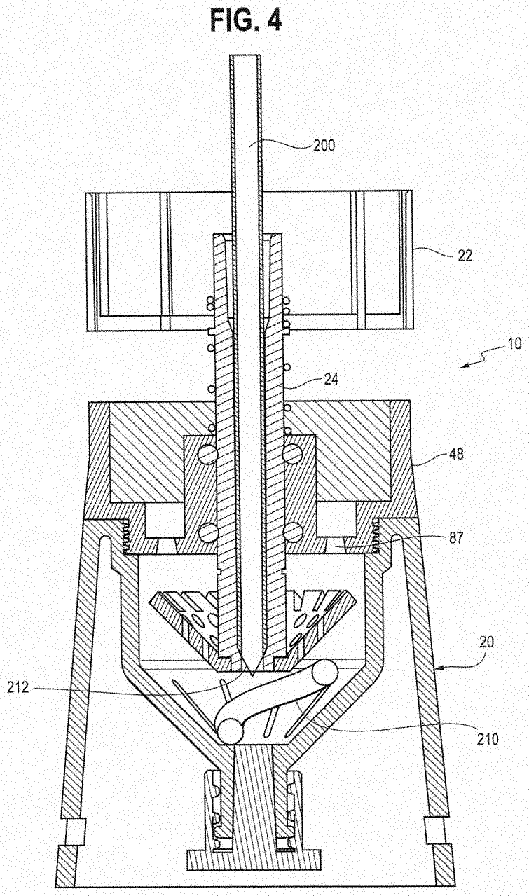

Referring now to FIGS. 2-12, the operation of the system 10 will be described. In the embodiments of the FIGS. 2-12, alternate structures and configurations of the apparatus are shown, by way of example and not of limitation, a threaded interface between the first or upper module 15 and the base module 20 formed by interacting threads 50, 51 formed in each of the modules respectively, and the upper and lower morselizing tools 105, 100 having cooperating angled configurations, it is to be understood that the function and operation herein described is applicable to all embodiments of the present invention.

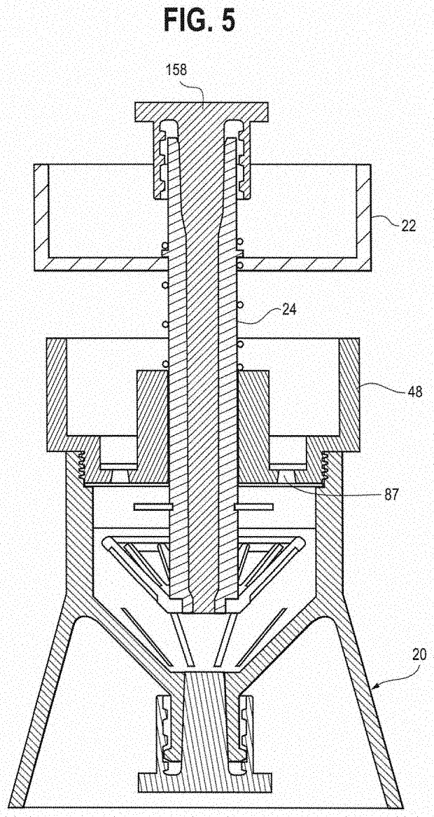

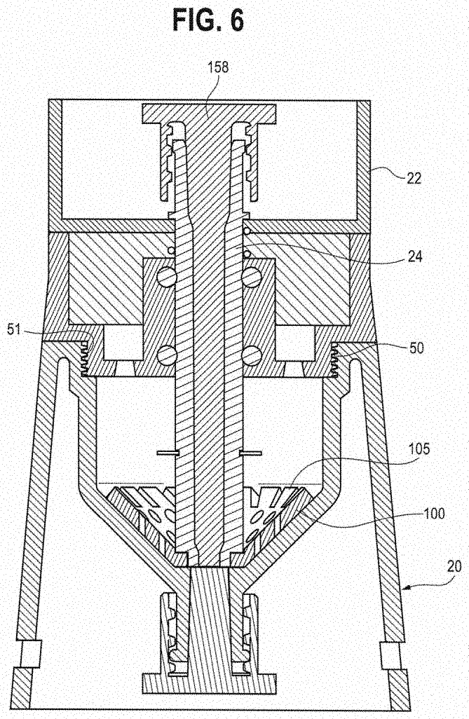

FIGS. 2-4 illustrate the steps of loading a sample of harvested biological material, for example bone core and MSC tissue, into the system. The tissue is harvested from a patient for diagnostic or treatment purposes or from a laboratory animal for research purposes via a Jamshidi needle or stylus 200 and syringe 205 as hereinabove described. After removing the plug 158, the Jamshidi needle and syringe are inserted into the cylindrical aperture 35 in the spindle 24 and the sample represented by the material 210 is injected into the receptacle 90 by depressing a plunger 207 in the syringe. A stop 212 positioned at the second end 28 of the spindle limits the insertion depth of the Jamshidi needle into the spindle. Following injection of the sample, the syringe and needle are removed, and the plug 158 is reinserted into the spindle 24 as shown in FIG. 5, and the knob 22 and upper grinding or morselizing tool or plate 105 are controllably urged at a preselected feed rate from the extended or open configuration into the depressed or closed configuration while the knob and upper grinding tool are simultaneously rotated at a preselected rotational speed as shown in FIG. 6. An exemplary processing protocol after loading the harvested material includes the following steps and parameters:

### Example

1. Lower the upper grinding tool to 1.5 mm above the lower grinding tool for 60 seconds.
2. Lower the upper grinding tool to 1.0 mm above the lower grinding tool for 60 seconds.
3. Lower the upper grinding tool to 0.75 mm above the lower grinding tool for 60 seconds.
4. Lower the upper grinding tool to 0.50 mm above the lower grinding tool for 60 seconds.
5. Lower the upper grinding tool to 0.25 mm above the lower grinding tool for 60 seconds.
6. Lower the upper grinding tool to 0.10 mm above the lower grinding tool for 30 seconds.
7. Lower the upper grinding tool to 0.05 mm above the lower grinding tool for 30 seconds.

The morselizing/grinding procedure is performed with the upper grinding tool being rotated at a constant rotation speed of 180 RPM. Upon completion of processing, the fluid suspension containing GAG is collected and residual bone fragments are left in the receptacle. However, it is to be understood that the upper grinding tool feed rate and rotational speed may be selectively varied independently depending upon harvested material and desired end product characteristics and properties to optimize the process efficiency and quality of the resultant product.

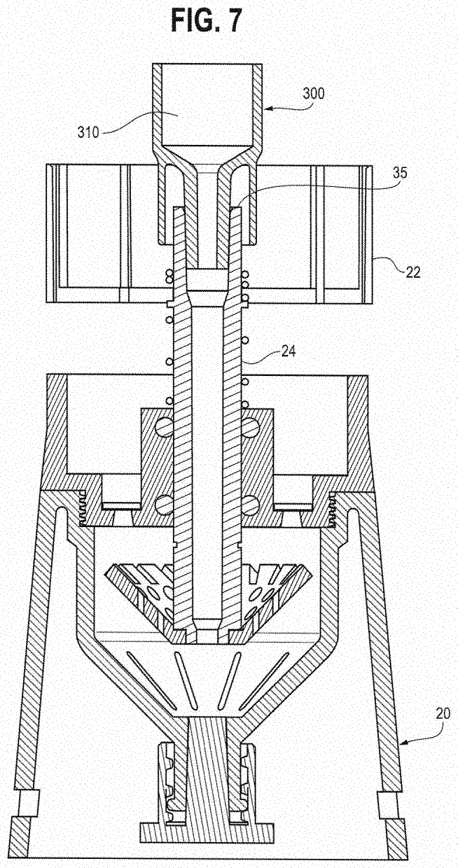

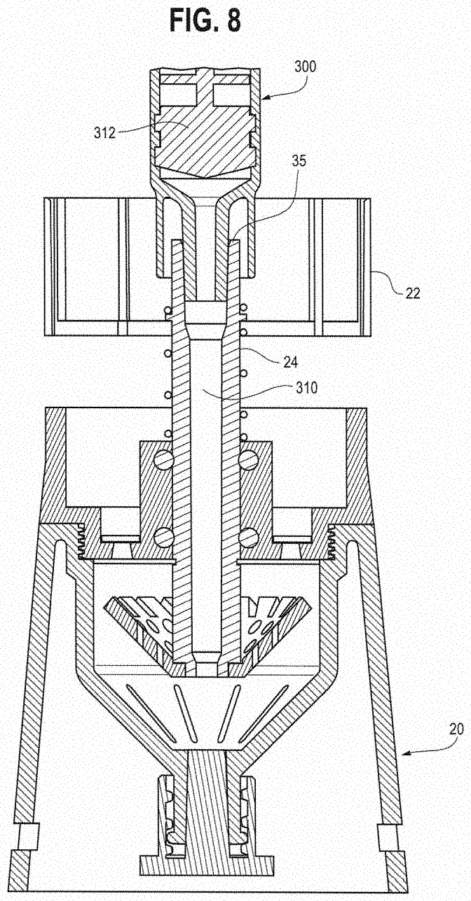

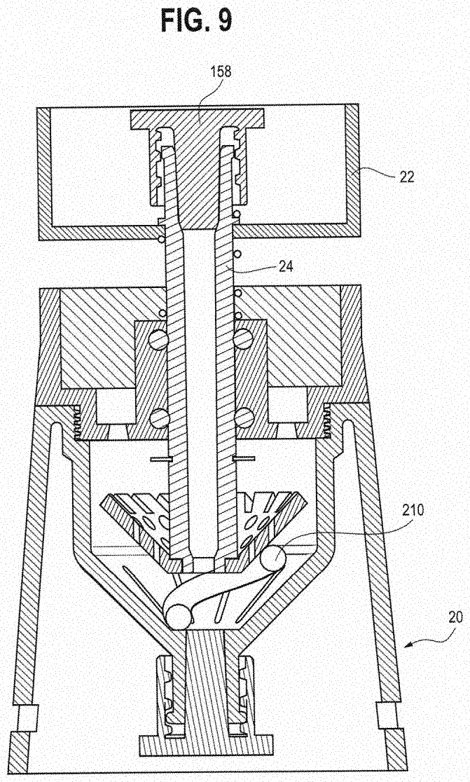

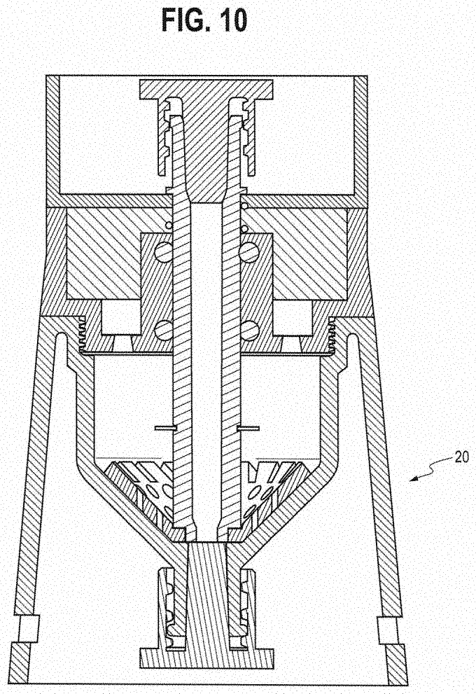

Additional washing or rinsing steps may be performed as needed during the morselizing process to enhance the purity and output quantity of the product, which are illustrated in FIGS. 7-9. The knob and upper grinding tool are withdrawn upwardly into the extended or open position, the plug 158 is removed, and a syringe 300 containing rinse or carrier fluid 310 is inserted into the cylindrical aperture 35 in the spindle 24. The rinse or carrier fluid is injected into the receptacle 90 by depressing a plunger 312 in the syringe (FIGS. 7 and 8). The rinse or carrier fluid may include by way of example bone marrow, blood, plasma, serum, saline, water, anticoagulant or other biologically compatible materials or a combination of the foregoing. However, is saline solution has been shown to produce the best results and is the preferred carrier fluid. Following each rinse step, the plug 158 is reinserted into the spindle aperture 35 (FIG. 9), the knob and upper grinding tool are controllably advanced from the extended or open position into the depressed or closed configuration and the morselizing and agitating process is repeated, as shown in FIG. 10.

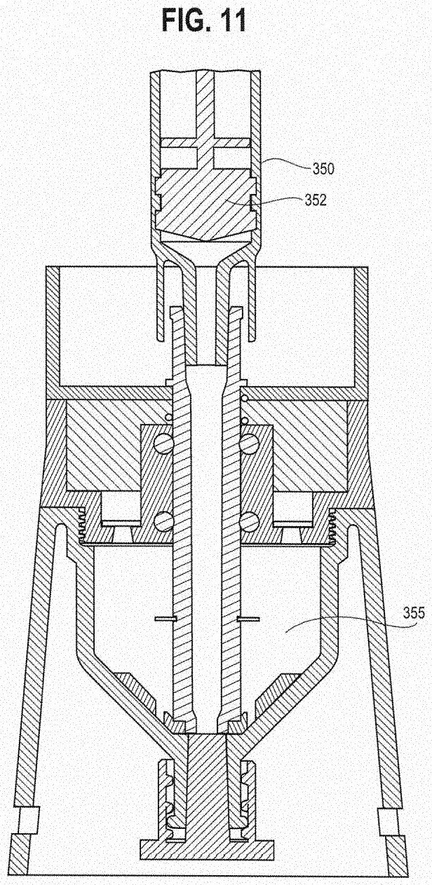

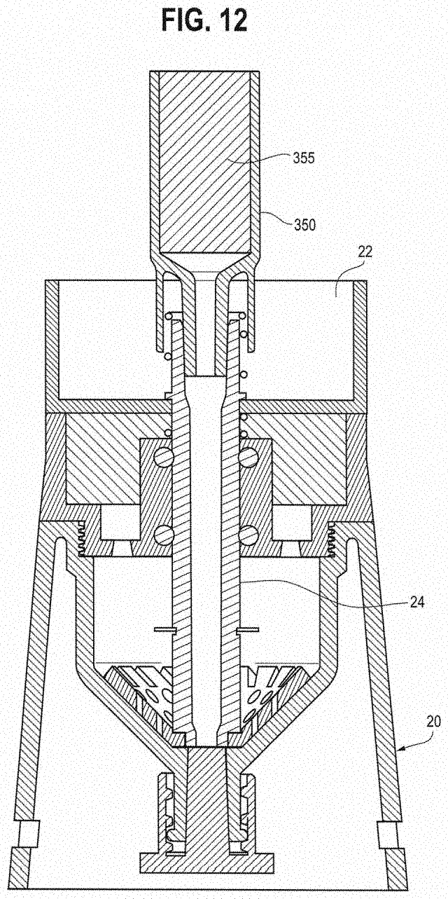

Upon completion of the processing operation, the product is harvested as illustrated in FIGS. 11 and 12. With the system in the closed or depressed configuration, the plug 158 is again removed, and a syringe 350 having a plunger 352 is inserted into the aperture 35. As the plunger is withdrawn, processed material/product 355 is drawn from the modular receptacle 90 into the syringe for use in an intended application. Thereafter, the modules may either be discarded or, alternatively washed and sterilized for subsequent use.

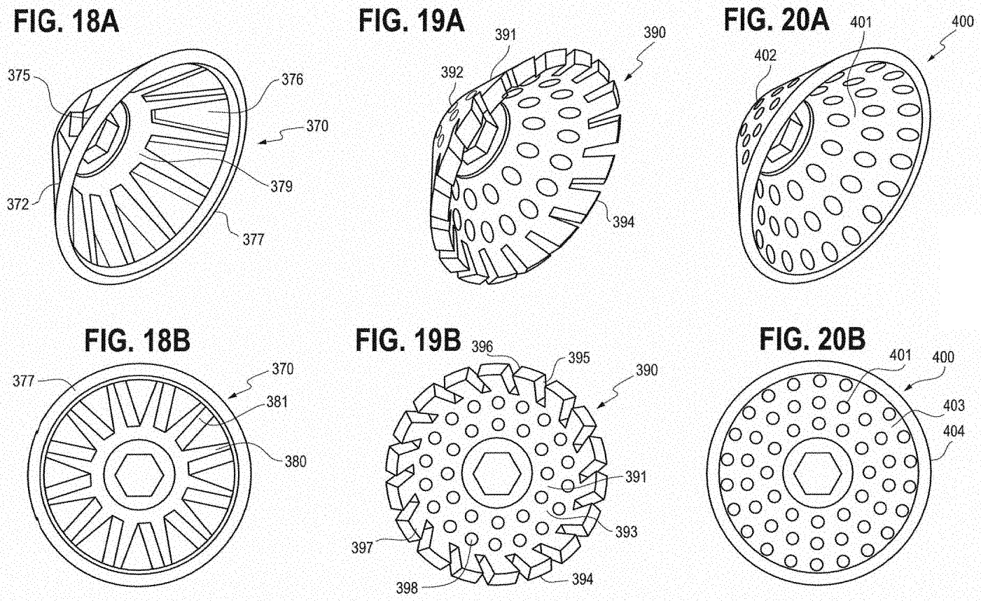

FIGS. 18.A-20. B illustrate alternate morselizing/grinding plate configurations. The design shown in FIGS. 18.A and 18.B is in the form of a blender blade structure having a conically shaped body 372, a first end 375 adapted to be operatively connected to the end 28 of a spindle 24 and a second open end 376 having a circular peripheral edge 377. A plurality of equally spaced apart blades 378 extend from and are secured at a first end 379 thereof to the end of the conically shaped body and are secured at a second end 380 to the circular peripheral edge 377. Each of the blades includes a tapered face 381 formed thereon which is structured and arranged to agitate the harvested material in the receptacle.

A hybrid morselizer plate design is 390 shown in FIGS. 19.A and 19.B. Morselizer 390 includes a conically shaped body 391, a first end 392 adapted to be operatively connected to the end 28 of a spindle 24 and a second open end 393 having a circular peripheral edge 394. A plurality of equally spaced apart slots 395 extend from the circular peripheral edge 394 to a preselected depth, each slot including oppositely disposed angled faces 395, 396 forming a finger or blade 397 therebetween. The conically shaped body has a plurality of apertures 398 extending therethrough adapted to further increase the agitation and mixing of harvested bone core material.

Yet another morselizer plate design is depicted in the embedment 400 of FIGS. 20.A and 20.B. Similar in configuration to the embodiment of FIGS. 19.A and 19.B, morselizer 400 includes a conically shaped body 401, a first end 402 adapted to be operatively connected to the end 28 of a spindle 24 and a second open end 403 having a continuous circular peripheral edge 404. The conically shaped body has a plurality of apertures 405 extending therethrough to adapted to further increase the agitation and mixing of harvested bone core material.

Human-machine interface (HMI) may provide an auditory, tactile, textual, or graphical view of system conditions or operations and may be configured to allow an operator to monitor or control various aspects of the system. Alternatively, the HMI may be configured to report the status of various aspects of the system or process.

Motion control components selected and configured for accurate position/velocity/torque capabilities operating in either open or closed loop mode and may include servo systems, stepper systems, gearboxes, linear motion slides and actuators and drive couplings. A closed loop servo system may comprise feedback devices at the motor shaft to verify or adjust the resulting motion and may be configured to receive a command signal(s) for step and direction, CW/CCW, A/B Quad, internal indexing (for example, via discrete input and communication port), torque and velocity (for example, via analog input) and may include a control mode for torque, velocity and position.

The bench top configuration and/or other aspects of the system may comprise various power sources, programmable controllers, microprocessors, transmitters, encoders, gauges, meters, buttons, switches, indicators, interlocks or sensors having discrete or analog outputs configured to receive, send or provide data for automating various aspects of the system or process including rotary encoders for detecting position and speed; sensors for detecting proximity (inductive, magnetic, capacitive, ultrasonic), presence or distance (photoelectric), pressure, temperature, level, flow, turbidity, electrical conductivity, contrast, color, light transmission at one or move wavelengths, current and voltage; laser sensors, fiber optic sensors, fork sensors (or slot sensors); and limit switches for detecting states such as presence or end-of-travel limits.

The bench top embodiment and/or other aspects of the system may comprise various analyzers (to detect, quantify, profile, to characterize or measure the modular container constituents), metrological or cytometric modules, probes, components, reagents, assays, instruments, and systems. On-board cytometry may provide for the measurement or determination of the characteristics of cells for closed loop machine feedback or for operator display and may measure cell size, cell count, cell morphology (shape and structure), cell cycle phase, DNA content, RNA content, and the existence or absence of specific proteins on the cell surface or in the cytoplasm or otherwise characterize and count certain cells (for example red blood cells). For example, image cytometers, flow cytometers, cell sorters, time-lapse cytometers, assays, ELISA (enzyme-linked immunosorbent assay), real-time PCR systems may be incorporated in the system. In an embodiment, sampling plumbing may fluidically connect the chamber with one or more analyzers to selectably sample the container constituents before, during or after processing. In another aspect, the modular container may have an auxiliary draw port for manual sampling, and the sample may be delivered to a dry western blotting system for rapid analysis to monitor protein levels of, e.g., a-SMA and calponin. In particular aspects, a sample may be taken from the container through the plumbing and delivered to a real-time PCR module of the system in order to examine gene expression to determine if the shear stress caused by the cycle of the processing has markedly changed (e.g., increased) the mRNA levels of, e.g., CD31 and VE-cadherin. Such monitoring of gene expression may provide data in a control loop to control processing duration, speed and other parameters desirable for a particular patient, application, or other concern. The system may pause processing during sample analysis before either continuing processing or stopping processing depending on the data from the analysis.

The system is configured to collect, handle, process and deliver quickly, accurately, reproducibly, and reliably the biologic mixture from and back to a patient for an autologous point-of-care procedure. The disposable module may be configured, programmed, and controlled in a particular manner such that the washing cycle may impart a range of shear stresses on the stem cells or other constituents of the tissue or carrier fluid. It has been demonstrated that particular levels of shear stress will direct stem cell development along certain developmental pathways. The disposable module and processing system may be designed to selectively, repeatably and controllably impart a range of shear stresses on the stem cells to encourage development along numerous pathways so that the stem cells can be used in multiple applications autologously in the patient's body (Shear stress magnitude is critical in regulating the differentiation of mesenchymal stem cells even with endothelial growth medium-Biotechnol Lett. 2011 December; 33(12):2351-9. doi: 10.1007/s10529-011-0706-5. Epub 2011 Jul. 31.), which is incorporated herein by reference in its entirety, also see (Response of mesenchymal stem cells to shear stress in tissue-engineered vascular grafts-Acta Pharmacol Sin. 2009 May; 30(5): 530-536, published online 2009 May 5. doi: 10.1038/aps.2009.40), which is incorporated herein by reference in its entirety. The shear stress or an average shear stress may be in the range of 0.5 a to 1.5 Pa, 5 Pa to 100 Pa or 200 to 500 Pa. A maximum shear stress or a maximum average shear stress may be limited to about 0.5 Pa, 1 Pa, 1.5 Pa, 5 Pa, 15 Pa, 50 Pa, 75 Pa, 100 Pa, 200 Pa, 500 Pa, 700 Pa, 2500 Pa or 3000 Pa.

For example, shear stress and duration of exposure to the shear stress may be controlled by the grinding/washing tooling portion rotational speed, geometry or other parameters to maintain cell viability while being sufficient to detach certain material, e.g. GAG, from the bony matrix of the tissue and while reducing the particulate size to a desirable level. The maximum shear stress may be from about 0.5 Pa to about 1.5 Pa for a relatively long exposure duration; on the other hand, for short duration exposure, the maximum shear stress may be from about 700 Pa to about 2,500 Pa.

As an example, the chamber may be filled with about 1.75 milliliters of carrier fluid (e.g., normal saline) and about 100 milligrams of tissue. The washing tooling portion rotational speed may be run at about 3,000 RPM for about 100 seconds.

As another example, the chamber may be filled with about 1.75 milliliters of carrier fluid (e.g., normal saline) and about 100 milligrams of tissue. The washing tooling portion rotational speed may be run at about 15,300 RPM for about 10 seconds.

As noted above, the carrier fluid may comprise saline, autologous blood plasma, an anticoagulant solution, sodium citrate 4% w/v, platelet rich plasma (PRP) (leukocyte-rich PRP (L-PRP), leukocyte reduced PRP (P-PRP; leukocyte reduced or pure PRP), leukocyte platelet-rich fibrin or pure platelet-rich fibrin), growth factors, cytokines, ligands, hormones, neurotransmitters, RNA, plasmids, synovial fluid, exosomes or cerebrospinal fluid. The chamber may be purged or infiltrated with oxygen, nitric oxide, carbon monoxide or other gases or gas mixtures prior to carrier fluid and or tissue delivery or following delivery or during processing, and the material in the receptacle may be centrifuged prior to extraction.

Results:

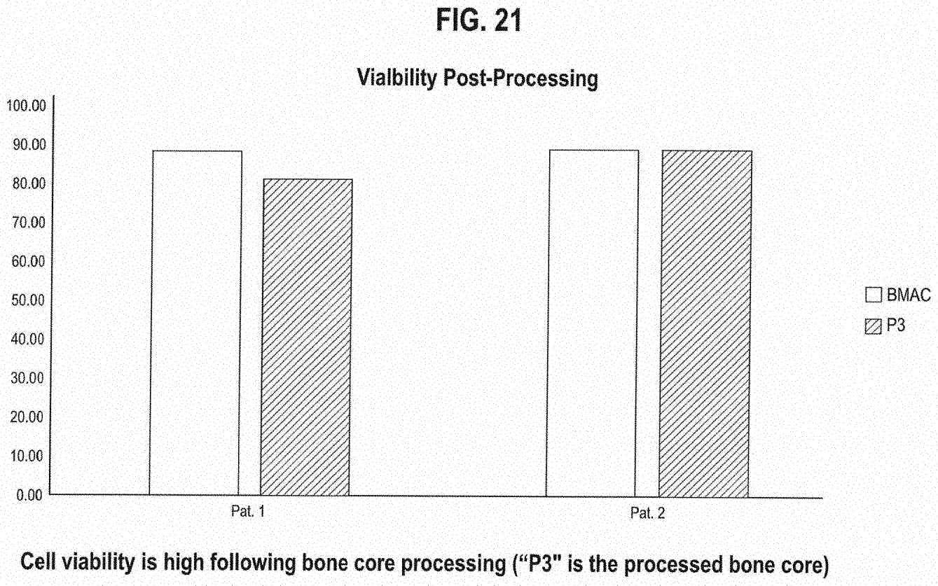

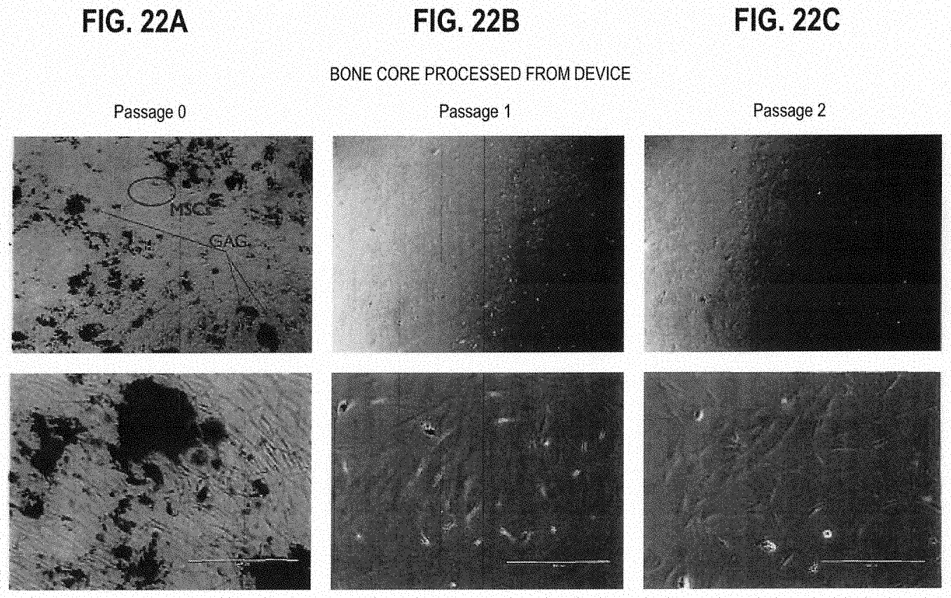

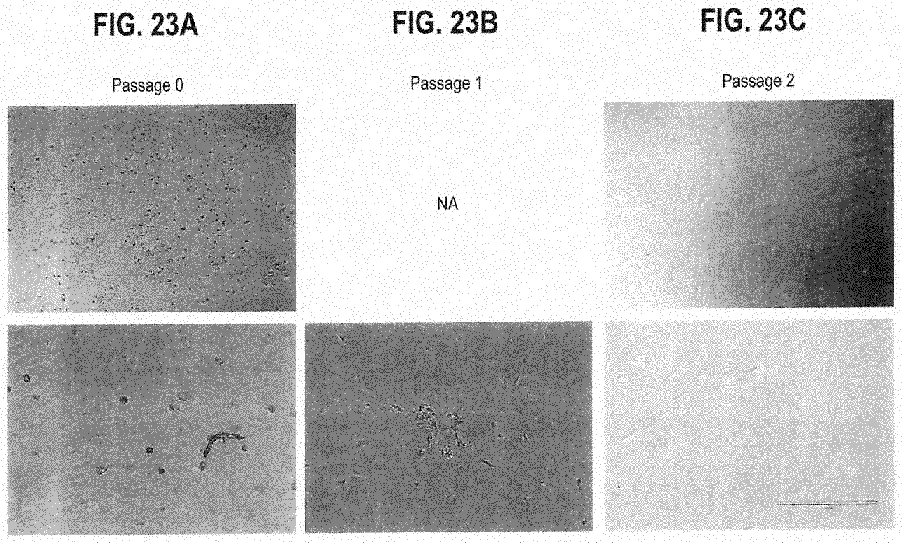

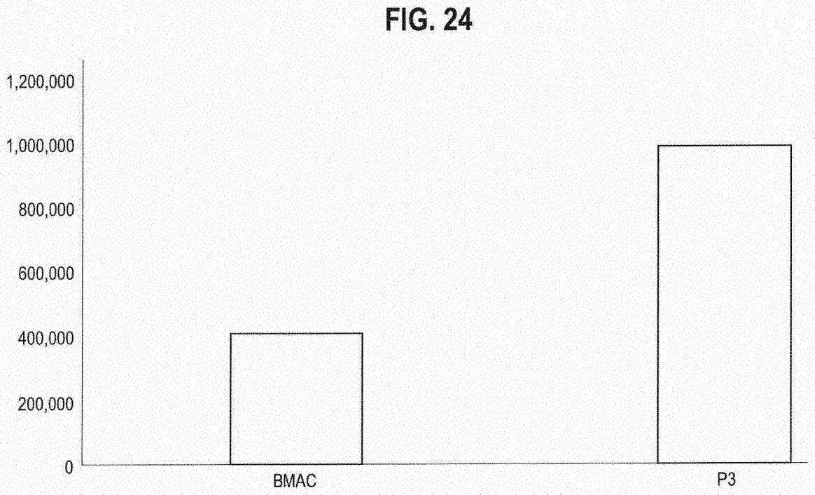

FIG. 21 illustrates the enhanced viability of two samples of bone core following processing in accordance with the present invention. Photomicrograph images of tissue at various stages of processing taken at 2×, 4× and 10× are shown in FIGS. 22.A-22.C for a first sample and FIGS. 23.A-23.C for a second sample illustrating the increased concentrations of MSC in the final product. This result is expressed graphically in the bar graph of FIG. 24 which illustrates the high density MSC growth in bone core post processing.

Changes may be made in the above methods and systems without departing from the scope hereof. It should thus be noted that the matter contained in the above description and/or shown in the accompanying figures should be interpreted as illustrative and not in a limiting sense. The following claims are intended to cover all generic and specific features described herein, as well as all statements of the scope of the present methods and apparatus, which, as a matter of language, might be said to fall there between.

What is claimed is:

1. A system for harvesting and processing a patient's mesenchymal stem cells (MSC's), the patient having a skeletal system having a cortical bone structure containing core bone material and marrow, blood, MSC and MSC-like cells, and physiological fluid, the system comprising:
a bone core harvesting instrument;
a modular receptacle or container adapted to receive the harvested bone core and any associated bone marrow, cells, and/or physiological fluid;
a securing device or base member adapted to receive and secure the modular receptacle or container;
a processing device adapted to process the harvested bone core in the modular receptacle or container; and
a device for extracting the processed harvested bone core from the modular receptacle or container.

2. The system of claim 1 wherein the bone marrow harvesting instrument is a Jamshidi needle (also known as a trocar or stylet), the Jamshidi needle be adapted to be movably deployed within an outer cannula, the needle being adapted to create an aperture in the patient's cortical bone structure at a preselected location and to collect core bone material and marrow, blood, MSC and MSC-like cells, and physiological fluid contained therein.

3. The system of claim 2 wherein the processing device includes a first module adapted to be operatively connected to and to cooperate with the base member for the processing of collected core bone material and marrow, blood, MSC and MSC-like cells, and physiological fluid.

4. The system of claim 3 wherein the first module includes a spindle have first and second ends, a handle operatively connected to a first end of the spindle, a morselizing tool or plate operatively connected to the second end, and a closure device including a first cylindrical body member adapted to secure the first module in sealing engagement with the base member.

5. The system of claim 4 wherein the base member includes a central axis, a tapered cylindrically shaped body having upper and lower open ends, a cylindrical wall portion extending circumferentially about the central axis intermediate the first and second open ends, a second cylindrical body member operatively connected to a bottom surface of the first cylindrical body member and extending downwardly therefrom into the base member.

6. The system of claim 5 wherein the modular receptacle is adapted to fit in releasable sealing engagement with the second cylindrical body member.

7. The system of claim 6 wherein the modular receptacle includes a closed bottom portion having an inner surface and a grinding or morselizing tool operatively connected to the inner surface.

8. A method for processing bone marrow comprising the steps of:
a. processing a tissue sample with a processing system, the system including modular receptacle having a first morselizing tool positioned therein, a spindle having a first and a second end having a second morselizing tool secured thereto, a body portion extending between the first and second ends, and a cannulation extending therethrough;
b. delivering a tissue sample into modular receptacle through the cannulation;
c. actuating the spindle at a preselected rotational speed;
d. advancing the morselizing tool at a preselected feed rate into operative engagement with the first morselizing tool for a preselected period of time;
e. extracting the processed tissue from the modular receptacle.

9. A method for preparing a therapeutic compound from a tissue sample, the method comprising:
a. obtaining a tissue sample with a harvesting tool,
b. delivering the tissue sample to a processing system, the system including an actuator assembly, a modular receptacle having a first morselizing tool positioned therein, a spindle having a first and a second end having a second morselizing tool secured thereto, a body portion extending between the first and second ends, and a cannulation extending therethrough;
c. actuating the spindle at a preselected rotational speed;
d. advancing the morselizing tool at a preselected feed rate into operative engagement with the first morselizing tool for a preselected period of time;
e. extracting the processed tissue from the modular receptacle; and
f. processing a therapeutic compound from the extracted tissue.
